(19) United States
(12) Patent Application Publication
(10) Pub. No.: US 2025/0082187 A1
(43) Pub. Date: Mar. 13, 2025

(54) Medical Imaging Device with Heat Dissipation and Method for Regulating Heat Generated within a Medical Imaging Device

(71) Applicant: KARL STORZ SE & Co KG, Tuttlingen (DE)

(72) Inventors: Lars Hedemann, Tuttlingen (DE); Jonas Forster, Tuttlingen (DE)

(73) Assignee: KARL STORZ SE & Co KG, Tuttlingen (DE)

(21) Appl. No.: 18/826,010

(22) Filed: Sep. 5, 2024

(30) Foreign Application Priority Data
Sep. 7, 2023 (DE) 102023124088.9

(51) Int. Cl. A61B 1/06; A61B 1/05; A61B 1/12

(52) U.S. Cl. CPC A61B 1/06; A61B 1/05; A61B 1/128

(57) ABSTRACT

A medical imaging device such as an endoscope or exoscope is presented. The device has a light source for illuminating a viewing area and an image sensor, and may include a shaft having a distal section, a proximal section, an end face arranged on the distal section. The light source and the image sensor are accommodated in a common compartment at the distal section of the shaft and an operating heat emitted by the light source and/or by the image sensor is dissipated by means of a heat sink. Furthermore, the invention relates to a method for controlling and/or regulating a medical imaging device in response to the generated heat exceeding a predetermined threshold.

### CROSS REFERENCE TO RELATED APPLICATIONS

This application claims priority to German Patent Application No. 102023124088.9, filed Sep. 7, 2023, and entitled, “Medizinische Bildgebungsvorrichtung, insbesondere Endoskop oder Exoskop, sowie Verfahren zum Steuern und/oder Regeln einer medizinischen Bildgebungsvorrichtung,” which is incorporated herein by reference.

### FIELD OF THE INVENTION

This invention relates to a medical imaging device and more particularly to an endoscope having a shaft, a light source for illuminating a viewing area, and an image sensor for detecting image light captured from the imaging area.

### BACKGROUND OF THE INVENTION

Medical imaging devices for capturing and processing images from an object field or imaging area include both endoscopes and exoscopes, Conventional endoscopes generally include a shaft, a light source for illuminating a viewing area and an image sensor for recording image light captured from the viewing area. While the invention will be primarily described throughout as relating to endoscopes, which include a shaft element which may extend into a an object under observation, such as a human body cavity, exoscopes generally do not include a shaft per se, however this invention applies generally to exoscopes as well as endoscopes, and the embodiments described herein are illustrative and one of skill in the art would easily understand how the described invention applies to exoscopes. The endoscopic shaft has a distal section, a proximal section, and a longitudinal axis running from the distal section to the proximal section. The shaft includes an end face arranged on its distal section and a shaft wall extending along the longitudinal axis with an outer surface facing an external environment. Certain endoscopes, often referred to as “chip-on-the-tip” (COTT) endoscopes generally include a light source and image sensor accommodated at a distal portion of the shaft. Associated with the light source and the image sensor is an operating heat emitted by these elements.

Conventional medical imaging devices such as endoscopes and exoscopes may employ a “heat pipe” as the heat sink within the shaft in order to dissipate operating heat from the light source and/or an image sensor away from the distal section, and various configurations are known in the art for coupling the light source and and/or the image sensor to the heat sink by means of a heat transfer element. These known methods however, often result in an arrangement where the light source and the image sensor are located in separate chambers within the distal head of the scope, which often leads to sealing problems. This can be particular problematic when sterilizing such medical imaging devices, as the failure of individual seals can lead to the ingress of hot steam, for example, as these seals are complicated and comprise several sealing levels.

What is needed is a medical imaging device, such as an endoscope or an exoscope, with improved heat dissipation and without the complexity of multiple seals each of which may be compromised.

### BRIEF DESCRIPTION OF THE INVENTION

As discussed above, each of the light source and the image sensor generate a heat associated with their operation. It is desirable that this generated heat be dissipated by means of a heat sink in the direction of a proximal region away from the distal tip of the endoscope or exoscope. The light source and the image sensor may be assigned a heat transfer element to conduct the operating heat from the light source and/or from the image sensor to the heat sink connected in a heat-conducting manner to the heat transfer element. The invention disclosed herein also relates to a method for controlling and/or regulating such a medical imaging device.

The present invention includes a shaft (when appropriate, for example, for endoscopic embodiments), a light source for illuminating a viewing area, and an image sensor for recording captured image light from the viewing area. The shaft includes a distal section, a proximal section, a longitudinal axis extending from the distal section to the proximal section, an end face arranged at the distal section and a shaft wall extending along the longitudinal axis with an outer surface facing the environment. The light source and the image sensor are accommodated in the distal section of the shaft, and an operating heat emitted by the light source and/or by the image sensor is dissipated by means of a heat sink in the proximal direction. The light source and the image sensor are assigned a heat transfer element to conducting the operating heat from the light source and/or the image sensor to the heat sink, wherein the heat transfer element has a first contact surface for contacting the light source and a second contact surface for contacting the image sensor. The light source and the image sensor transfer a respective operating heat to the heat transfer element by means of contact with their respective contact surfaces such that the operating heat of the light source and the operating heat of the image sensor are conducted to the heat sink by means of a common heat transfer element. The heat transfer element, which contacts both the light source and the image sensor with the first contact surface and the second contact surface respectively, enables the light source and the image sensor to be disposed in a common compartment, whereby the common heat transfer element can then transfer and dissipate corresponding operating heat to the heat sink. This single common compartment may hermetically sealed, decreasing, thereby, the number and complexity of seals prone to failure that are present in conventional systems.

The following terms are used throughout the specification.

A “medical imaging device” can be any technical and/or electrical device that is suitable for recording, processing, and/or transmitting an image of a viewing area of a medical environment. The image data may be displayed on a screen. Example such medical imaging devices are endoscopes and exoscopes. An “endoscope” is an imaging device, with a narrow and elongated shaft suitable for inserting into a cavity or through a usually small opening or incision in order to image areas arranged within the cavity and/or behind the small opening. Endoscopes that include the image sensor in the area of a distal tip are also referred to as “chip-on-the-tip” endoscopes. Such endoscopes are inserted into the body during medical procedures and used to image the viewing area within. An “exoscope” refers to a comparable medical imaging device that is used outside the body. It should further be noted the term “endoscope” can refer to thin, insertable portion of such a medical imaging device, but is also used as a term for an overall imaging system. Such endoscopic systems can be provided with further devices, for example a cable guide, further sensors and/or a display device for displaying image information on an external monitor. The terms “endoscope” and “endoscope system” are often used interchangeably and sometimes synonymously. The same applies to exoscopes.

A “shaft” refers to an elongated and very narrow area of the imaging device, in particular of an endoscope, which is often made of metal and connects, for example, a handle used to grip an endoscope to the actual distal tip section. Such a shaft can, for example, be formed as a tubular body made of stainless steel. In other embodiments, the shaft is flexible.

A “light source” is, for example, an LED, an incandescent lamp, or another light-emitting device. In the context of the invention, this also specifically refers to light sources that are equipped with an arrangement of an LED, several LEDs, or other light-emitting devices positioned in the distal section of the medical imaging device. The light source can direct illumination light to a desired location in the viewing area, thereby illuminating the viewing area with broad spectra white light or, alternatively light of one or more specific spectral bandwidths. It should be noted that the light source can also consist of several partial light sources, for example differently colored LEDs that are configured to illuminate the viewing area with different color spectra and/or provide illumination at different angles. Light sources can also be arranged at a spatial distance and/or angle from each other.

A “viewing area” describes the region, the volume, or a surface area which is under observation and to be viewed by means of the medical imaging device, and from which a corresponding image is to be generated or imaged. Such a viewing area is, for example, an organ, a bone, a lumen, a section of a human or animal body, or another area of interest for a corresponding observation.

An “image sensor” is, for example, an electronic chip or other similar device by means of which an image from the viewing area can be recorded along an optical path and converted into electronic signals, as are known in the art. For example, such an image sensor is a CCD chip or a comparable electronic component, such as a CMOS sensor. In relation to the invention, the image sensor is arranged together with the light source in the distal section of the medical device. It should also be noted that the “image sensor” as defined herein need not be limited to a single chip but can also be an arrangement of several and/or different image sensors, which may be employed to view the viewing area from multiple perspectives and/or in relation to different wavelengths of light.

A “distal section” here refers to an area of the shaft, when used in relation to an endoscope or a distal portion when used in relation to an exoscope, that is distanced from and faces away from the operator or user of the medical imaging device. In contrast, a “proximal section” refers to the section of the medical imaging device and/or the shaft facing an operator of the medical imaging device. A “longitudinal axis” is a connecting axis between the distal section and the proximal section, whereby this longitudinal axis can also be referred to as the main axis of the medical imaging device and does not necessarily have to be mathematically precisely defined. Rather, the longitudinal axis represents the essential axis of the medical imaging device, for example, along the shaft.

An “end face” in this context refers to a surface or plane arranged at the distal section and terminating the shaft and/or the medical imaging device in the direction of the viewing area. The end face can be arranged orthogonally to the longitudinal axis or at an angle to the longitudinal axis. For example, a normal of the front surface can be inclined by 30° to the longitudinal axis to realize a viewing direction that deviates by 30° from the longitudinal axis. Other angles are possible in this context. If, for example, several partial light sources and/or several image sensors are present, the end face refers to a surface aligned in a main illumination direction and/or in a main viewing direction.

The shaft itself includes an outer a “shaft wall”, which physically defines the radial extent shaft in the direction of an environment and, for example, in the case of a tubular shaft, forms a cylindrical surface, whereby the associated cylinder has a central axis congruent with the longitudinal axis. In some areas, the shaft wall will not be uniformly consistent along its length but may have connecting surfaces or constrictions. The shaft wall may be an element of the sheath.

“Operating heat” refers to the increase in temperature caused by the light source and/or the image sensor, which is generated and/or emanated due to, for example, a power loss of the light source and/or the image sensor. The operating heat is transmitted to a “heat sink”, i.e. to an element with, preferably, a high thermal conductivity and/or high heat capacity. In some instances, such a heat sink can be designed as a “heat pipe”, i.e. as a thermodynamically active conductive element for conveying heat from a heat source in the direction of a heat-absorbing structure of the medical imaging device. The operating heat is dissipated from the distal area of the shaft where the light source and the image sensor are located towards the proximal area of the shaft.

A “heat transfer element” is an element formed from, for example, a thermally conductive material, to which the light source and/or the image sensor are thermally connected in order to absorb corresponding operating heat initially into the heat transfer element and to transmit the operating heat to the heat sink. According to the present invention, the heat transfer element has a first contact surface and second contact surface. Each respective “contact surface” is designed for, generally, physically and thermally connecting the heat transfer element to the light source and the image sensor. For example, there is a correspondingly created metallic surface on the heat transfer element which is designed to contact one side of the light source or to a rear side of the image sensor, so that, for example, the highest possible surface area contact is achieved. The “contact” is the physical contact between the light source and the image sensor on the respective contact surface. It should be mentioned here that in an arrangement with several partial light sources and/or several image sensors, respective contact surfaces or further contact surfaces may be present for physically contacting the partial light sources and/or the image sensors. One of the innovations of the present invention is that the respective light source or the respective light sources and/or the image sensor or the respective image sensors are thermally coupled to a common heat transfer element in a physically contacting manner.

Thereby, the light source and the image sensor can be arranged in a common compartment positioned in the distal section with the heat transfer element being arranged at least partially in the common compartment.

The common “compartment” describes a volume that is sealed by sealing elements and in which the light source and the image sensor are arranged together in order to reduce the number and complexity of corresponding sealing elements that would be required for separate compartments, as is traditionally done. For example, the heat transfer element can be arranged in such a way that it extends at least partially, including while the first contact surface and the second contact surface, into the common compartment and is thus arranged at least partially in the common compartment. Alternatively, the heat transfer element can be arranged completely in the common compartment so that, for example, the heat sink is guided through a boundary of the compartment.

In one embodiment, the light source and the image sensor are arranged in a common plane; additionally or alternatively, a lens can be arranged in the end face for distal closure of the common recording space. Such a lens arranged in the distal face may be referred to as an end lens. This configuration makes it possible to achieve a particularly compact arrangement, especially in cases where the medical imaging device has an inclined front surface.

A “common plane” here refers to a mathematically essentially congruent plane in which both the light source and the image sensor are arranged with, for example, a respective component-related reference plane.

A “cover lens” refers to an optical boundary in the form of a cover glass that also seals the distal section of the medical imaging device.

In order to simplify the manufacture of the heat transfer element, the first contact surface and the second contact surface or other contact surfaces may be arranged essentially parallel to each other, with the end face and the contact surface being inclined from an end face plane arranged orthogonally to the longitudinal axis.

In one embodiment, the first contact surface and the second contact surface or other contact surfaces are arranged at an angle to one another, with the first contact surface or the second contact surface being arranged parallel to the end face and the respective other contact surface being arranged essentially parallel to the longitudinal axis.

With this design of the heat conducting element, the light source and the image sensor can be arranged on different contact surfaces in different geometric configurations in a correspondingly simple production-related manner.

In some embodiments, light source or the image sensor is arranged on the first contact surface parallel to the end face and the image sensor or the light source is arranged parallel to the longitudinal axis, wherein a prism and/or a mirror is associated with the respective image sensor arranged parallel to the longitudinal axis or the respective light source arranged parallel to the longitudinal axis, wherein a deflection of light between the end face and the image sensor or the light source is achieved by means of the prism and/or by means of the mirror.

By arranging the light source or the image sensor “upright”, i.e. parallel to the front surface, and the respective other element “horizontally” parallel to the longitudinal axis a particularly compact design of the medical imaging device is possible, particularly when a very narrow shaft is required.

A “prism” is an optical component in the form of a geometric body that can be used for various optical effects, such as the deflection of a beam of light within the prism. Total internal reflection on an inner surface of the prism can also be used to achieve a mirror-like deflection of a light path. In contrast, a “mirror” is an optically smooth surface that enables the reflection of light and thus the deflection of a light path, for example from a light beam incident along the longitudinal axis in the direction of an image sensor arranged “horizontally” in the shaft.

In one embodiment, the heat transfer element has a connection area facing the operating heat source and a discharge area facing the heat sink, the connection area being tapered relative to the shaft diameter and the discharge area being widened relative to the connection area, so that the light source or the image sensor can be arranged in a region of the shaft where the cross-section is smaller than that of the connection area of the discharge area in contact with the heat sink. Thus, a particularly narrow medical imaging device with a particularly narrow shaft diameter can be achieved.

The “connection area” is arranged in the distal section of the shaft, while the “dissipation area” serves to dissipate the operating heat in the direction of the heat sink and is arranged more proximally. “Tapered” refers to a design with a reduced cross-section at one area as compared to another area. Thus, for example, a stepped design of the heat transfer element may be achieved by means of the tapered connection area being widened, i.e. having a larger cross-section at the dissipation area. This creates an installation cross-section for a light source or image sensor, for example, by means of the tapered connection area.

In order to improve the heat transfer between the heat transfer element and the heat sink, the heat transfer element may have a depression in the discharge area that extends along the longitudinal axis to accommodate the heat sink.

Such a “depression” can, for example, be designed as a hole or other material recess into which the heat sink can be inserted thereby creating a larger contact surface area and improving heat transfer between the heat sink and the heat transfer element.

In one embodiment, the heat sink is formed by means of the shaft wall and/or by means of a heat pipe arranged along the longitudinal axis.

In some embodiments, the shaft wall can be used, at least in part, to dissipate heat, with a heat capacity inherent to the shaft wall. A “heat pipe” can also be used to dissipate heat. Such a heat pipe uses capillary effects and phase transitions of a cooling medium present in the heat pipe to transport heat without mechanical drive. The heat transport capacity of such a heat pipe generally far exceeds the thermal conductivity of, for example, a solid copper rod. In a further aspect, the problem of unwanted heat present in the distal region of the medical imaging device is solved by a method for controlling and/or regulating a medical imaging device according to one of the aforementioned embodiments with a method. An operating temperature of the light source and/or an operating temperature of the image sensor is determined by means of a first temperature sensor arranged at the light source or by means of a second temperature sensor arranged at the image sensor. The determined operating temperature is compared with a reference temperature so that a difference between the respective operating temperature and the reference temperature is determined. The light output of the light source is controlled or regulated and/or an image output of the image sensor based on the determined differential temperature so that the medical imaging device is controlled and/or regulated based on the operating temperature.

Thus the operating temperature of a medical imaging device, in particular an endoscope or an exoscope, can be controlled by means of a temperature sensor present, for example, in the light source or a partial light source or in the image sensor or in an image sensor, whereby, for example, a common maximum temperature is used to reduce the light output of the light source if, for example, the operating temperature of the image sensor exceeds a corresponding permissible reference temperature.

### BRIEF DESCRIPTION OF THE DRAWINGS

The invention is further explained by the following exemplary descriptions of the particular embodiments.

FIG. 1 is a schematic representation of a chip-on-the-tip endoscope in an isometric view.

FIG. 2A is a schematic representation of a tip of the endoscope of FIG. 1 in a sectioned side view.

FIG. 2B is a schematic, partially transparent representation of the tip of the endoscope of FIG. 1 in an isometric view.

FIG. 2C is a cutaway view of a heat sink of the endoscope of FIG. 1 in an isometric view.

FIG. 3 is a schematic sectional view of an alternative embodiment of the tip of an endoscope.

### DETAILED DESCRIPTION OF THE VARIOUS EMBODIMENTS

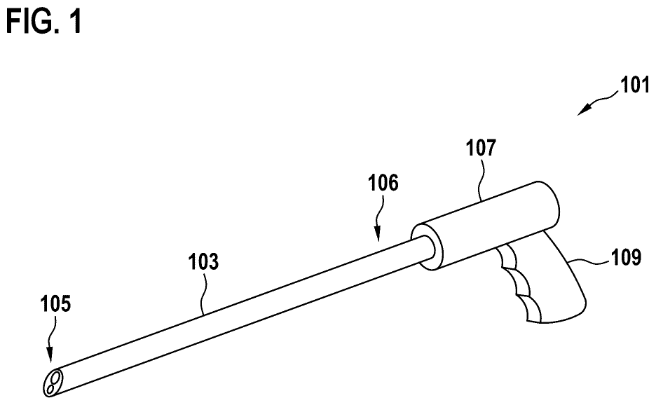

FIG. 1 shows endoscope 101 including a shaft 103, which is elongated and narrow in the form of a stainless steel tube. The shaft 103 has a tip 105 in a distal area. In a proximal region 106, a handle 107 including a gripping element 109 is arranged. The endoscope 101 can be guided by an operator using the handle 109. The distal tip 105 can be inserted into an area to be observed. The handle 107 may also contain operating elements and controls (such as buttons, triggers, joysticks, which are not described in detail here.

The shaft 103 includes a sheath 121 which, in the present example embodiment, is formed as a stainless steel tube. The sheath 121 encloses the illuminating and imaging components in the distal tip 105 and is described as below. The shaft wall may be an element of the sheath, that is the outer surface of the sheath, exposed to the environment, may be the shaft wall.

An image sensor 131 and a light source 133, such as an LED, are arranged in the tip 105. The image sensor 131 generates an image of a viewing area by means of a lens 171 and an optical system 173. The image sensor 131, the lens 171 and the optics 173 are separated from an environment by a cover glass 151, designated herein as the imaging cover glass, but it should be understood that the imaging cover glass 151 does not necessarily perform any optical manipulations. In some embodiments, however, the imaging cover glass may comprise an objective lens, while in other embodiments it is simply an optically transparent medium such as a glass plate. The LED 133 has a cover glass 153, referred to herein as an illumination cover glass. The imaging cover glass 151 and the illumination cover glass 153 are arranged parallel to a frontal plane 193 of the tip 105, the frontal plane 193 being arranged at an angle 195 relative to a longitudinal axis 191 along the shaft 103. In the embodiment shown in FIG. 1, the angle 195 is 30°, however in other embodiments it can be larger or smaller In some embodiments the angle is zero. Thus, the imaging cover glass 151 and the illumination cover glass 153 are arranged in an end face 194 of the tip 105, wherein the end face 194 is inclined along the angle 195 with respect to the shaft 103. The components are held in position in an interior 163 in the tip 105 by a spacer 161.

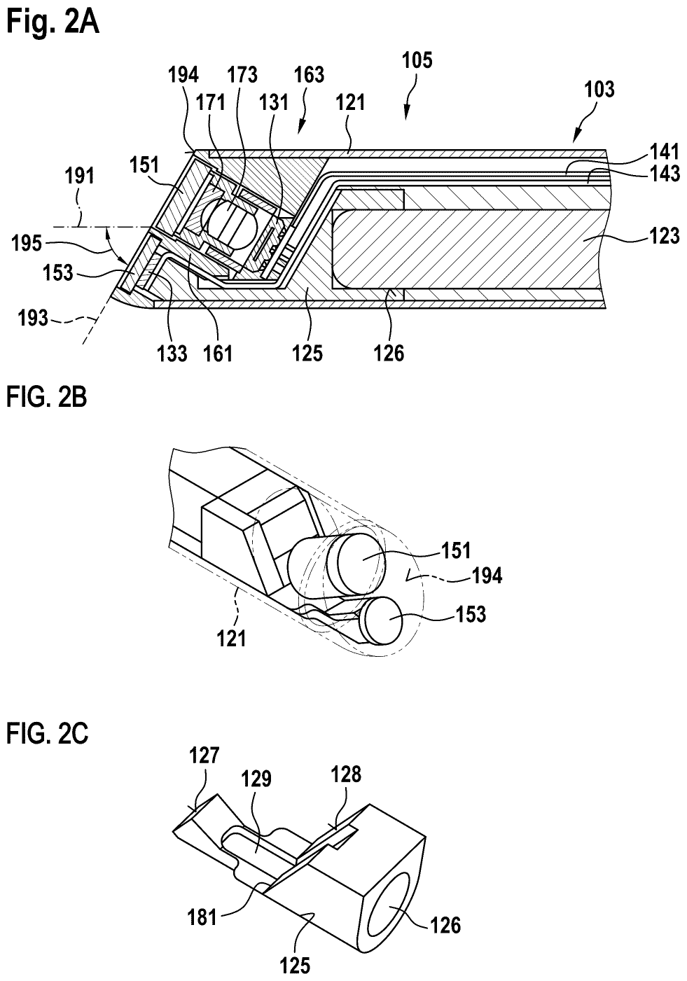

As shown in FIG. 2A, the LED 133 and the image sensor 131 are arranged near a heat sink 125. As most clearly seen in FIG. 2C, the heat sink 125 has a contact surface 127 for the LED 133 and a contact surface 128 for the image sensor 131. Thus, the operating heat generated by the LED 133 and the image sensor 131 is introduced via thermal connection into the heat sink 125.

The heat sink 125 is configured such that a distal section in the direction of the contact surface 127 is slim, whereas an area supporting the contact surface 128 is widened relative to the slim area. Further, in the embodiment shown, the heat sink 125 has a bore 126 into which a heat pipe 123, or another heat dissipating element, may be inserted. Heat dissipated by the LED 133 and the image sensor 131 through the heat sink 125 is thus transferred to the heat pipe 123 via contacting therewith and dissipated in the proximal direction, towards the handle 107.

A pocket 129 within the heat sink may be used to hold a circuit board 143, that is electrically connected to the LED 133, and may supply power thereto. Furthermore, a circuit board 141 is provided for making electrical contact with the image sensor 131. Both circuit boards 141 and 143 are designed as flexible circuit boards and are, in some embodiments, electrically connected to the handle 107 in order to electrically supply and control the LED 133 and the image sensor 131. These circuit boards may also carry image, temperature, and other information back to the handle region, which may, in turn, be connected to a display or camera control unit (CCU).

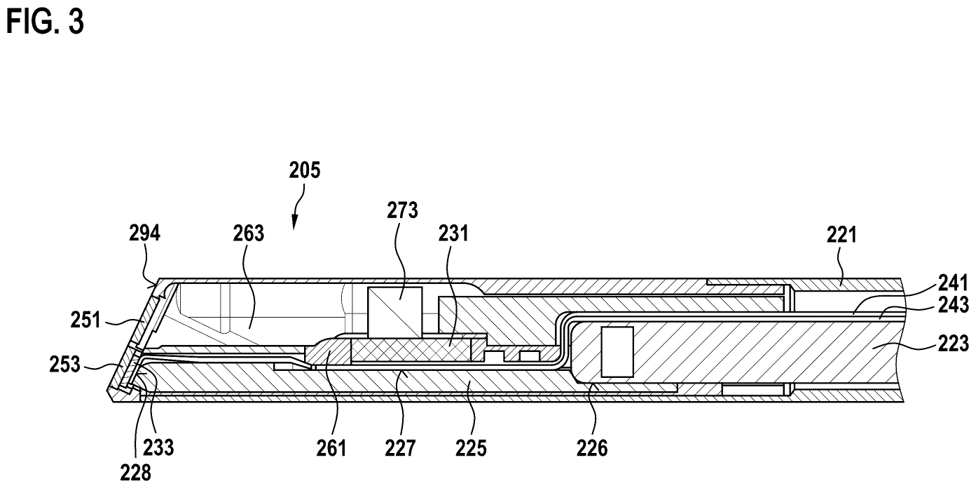

An alternative tip configuration 205 is shown in FIG. 3. This alternative embodiment of the endoscope 101 includes the tip configuration arranged within sheath 221. As in the embodiment described above, a heat pipe 223 is used to dissipate heat from an LED 233 and an image sensor 231. A heat sink 225 with a connection surface 226 is connected to the heat pipe 223. The heat sink 225 is elongated and arranged along a longitudinal axis of the sheath 221 and contacts the LED 233 with a contact surface 228. The contact surface 228 is arranged inclined parallel to an inclined end face 294. In the end face 294, as in the previous example, an imaging area is covered by means of an imaging cover glass 251 and the LED 233 is covered by means of an illumination cover glass 253.

In this embodiment, the image sensor 231 is arranged “horizontally”, i.e. parallel to a longitudinal axis of the sheath 221. Light information entering through the imaging cover glass 251 is deflected by 90° along an interior space 263 and through a prism 273 onto the image sensor 231.

During operation, some embodiments of the endoscope 101, either with tip 105 or with tip 205, a temperature sensor within the image sensor is used to monitor an overall temperature in the respective tip. If overheating of the image sensor 231 is detected, such overheating determined, for example, by noting that the temperature at the image sensor has exceeded a pre-set limit temperature, the power of the respective LED may be reduced or other corrective action may be taken.

The foregoing has outlined rather broadly the features and technical advantages of the invention in order that the detailed description of the invention that follows may be better understood. It should be appreciated by those skilled in the art that the conception and specific embodiments disclosed may be readily utilized as a basis for modifying or designing other structures for carrying out the same purposes of the invention. It should also be realized by those skilled in the art that such equivalent constructions do not depart from the scope of the invention as set forth in the appended claims.

Although the invention and its advantages have been described in detail, it should be understood that various changes, substitutions and alterations can be made herein without departing from the scope of the invention as defined by the appended claims. The combinations of features described herein should not be interpreted to be limiting, and the features herein may be used in any working combination or sub-combination according to the invention. This description should therefore be interpreted as providing written support, under U.S. patent law and any relevant foreign patent laws, for any working combination or some sub-combination of the features herein.

Moreover, the scope of the present application is not intended to be limited to the particular embodiments of the process, machine, manufacture, composition of matter, means, methods and steps described in the specification. As one of ordinary skill in the art will readily appreciate from the disclosure of the invention, processes, machines, manufacture, compositions of matter, means, methods, or steps, presently existing or later to be developed that perform substantially the same function or achieve substantially the same result as the corresponding embodiments described herein may be utilized according to the invention. Accordingly, the appended claims are intended to include within their scope such processes, machines, manufacture, compositions of matter, means, methods, or steps.

### LIST OF REFERENCE SYMBOLS

101 Endoscope
103 Shaft
105 Distal tip
106 Proximal region
107 Handle
109 Grip
121 Sheath
123 Heat pipe
125 Heat sink
126 Bore
127 Contact surface
128 Contact surface
129 Pocket
131 Image sensor
133 LED
141 Circuit board
143 Circuit board
151 Imaging cover glass
153 Illumination cover glass
161 Spacer
163 Interior
171 Lens
173 Optics
191 Longitudinal axis
193 Frontal plane
194 End face
195 Angle
205 Tip
221 Sheath
223 Heat pipe
225 Heat sink
226 Connection surface
227 Contact surface
228 Contact surface
231 Image sensor
233 LED
241 Circuit board
243 Circuit board
251 Imaging cover glass
253 Illumination cover glass
261 Spacer
263 Interior space
273 Prism
294 End face

1. A medical imaging device, comprising
a shaft comprising a distal tip, a proximal region, a longitudinal axis extending from the distal tip to the proximal region, an end face, and a shaft wall extending along the longitudinal axis and facing an environment;
a light source for illuminating a viewing area, wherein the light source is accommodated in the distal portion of the shaft;
an image sensor for recording image light captured from the viewing area, wherein the image sensor is accommodated in the distal portion of the shaft;
a common heat transfer element with a first contact surface and a second contact surface; and
a heat sink positioned proximally to the distal portion of the shaft;
wherein the light source and the image sensor are respectively thermally connected to the first and second contact surface of the common heat transfer element, and an operating heat generated by the light source and/or by the image sensor is dissipated toward the proximal region to the heat sink by the common heat transfer element.

2. The medical imaging device of claim 1, wherein the light source and the image sensor are arranged in a sealed common compartment in the distal portion of the shaft, and wherein the heat transfer element is arranged at least partially in the common compartment.

3. The medical imaging device of claim 2, wherein the light source and the image sensor are arranged in a common plane.

4. The medical imaging device of claim 3, wherein a single cover glass is arranged in the end face for distally terminating the common compartment.

5. The medical imaging device of claim 3, wherein an imaging cover glass and an illuminating cover glass are arranged in the end face and distally terminate the common compartment.

6. The medical imaging device of claim 1, wherein the first contact surface and the second contact surface are arranged substantially parallel to each other, and wherein the end face and the contact surfaces are arranged inclined from an end plane arranged orthogonal to the longitudinal axis.

7. The medical imaging device of claim 1, wherein the first contact surface and the second contact surface are arranged at an angle to each other, wherein the first contact surface or the second contact surface is arranged parallel to the end face, and wherein the respective other contact surface is arranged substantially parallel to the longitudinal axis.

8. The medical imaging device of claim 7 further comprising a prism or mirror, and wherein the light source or the image sensor is arranged on the first contact surface parallel to the end face and the image sensor or the light source is arranged parallel to the longitudinal axis, where the prism is configured to deflect the image light between the end face and the image sensor.

9. The medical imaging device of claim 7 further comprising a prism or mirror, and wherein the light source or the image sensor is arranged on the first contact surface parallel to the end face and the image sensor or the light source is arranged parallel to the longitudinal axis, where the prism is configured to deflect an illumination light between the light source and the end face.

10. The medical imaging device of claim 1, wherein that the heat transfer element has a connection region directed toward the operating heat and a discharge region directed toward the heat sink, where the connection region is tapered relative to a shaft diameter and the discharge region is widened relative to the connection region.

11. The medical imaging device of claim 1, wherein the heat transfer element has a depression at the discharge region, the depression extending along the longitudinal axis and receiving at least a portion of the heat sink.

12. The medical imaging device of claim 10, wherein the heat transfer element has a depression at the discharge region, the depression extending along the longitudinal axis and receiving at least a portion of the heat sink.

13. The medical imaging device of claim 11, wherein the depression comprises a shared heat transfer surface in thermal contact with the heat sink, and wherein said heat transfer surface includes a surface parallel to the longitudinal axis.

14. The medical imaging device of claim 12, wherein the depression comprises a shared heat transfer surface in thermal contact with the heat sink, and wherein said heat transfer surface includes a surface parallel to the longitudinal axis.

15. The medical imaging device of claim 1, wherein the heat sink comprises of the shaft wall.

16. The medical imaging device of claim 1, wherein the heat sink comprises a heat pipe arranged along the longitudinal axis.

17. A method for controlling and/or regulating a medical imaging device, comprising the steps of
providing a shaft comprising a distal tip, a proximal region, a longitudinal axis extending from the distal tip to the proximal region, an end face, and a shaft wall extending along the longitudinal axis and facing an environment;
providing a light source for illuminating a viewing area, wherein the light source is accommodated in the distal portion of the shaft;
providing an image sensor for recording image light captured from the viewing area, wherein the image sensor is accommodated in the distal portion of the shaft;
providing a common heat transfer element with a first contact surface and a second contact surface;
providing a heat sink positioned proximally to the distal portion of the shaft;
thermally connecting the light source and the image sensor respectively to the first and second contact surfaces of the common heat transfer element; and
dissipating an operating heat generated by the light source and/or by the image sensor toward the proximal region to the heat sink via the common heat transfer element.

18. The method of claim 17, comprising the further steps of
determining an operating temperature of the light source or an operating temperature of the image sensor by means of a first temperature sensor arranged in the light source or by means of a second temperature sensor arranged in the image sensor;
comparing the determined operating temperature with a reference temperature to determine a differential temperature; and
controlling or regulating a light output of the light source and/or an image output of the image sensor on the basis of the determined differential temperature.

19. The method of claim 18, wherein the light output or the image output is regulated such that the medical imaging device the differential temperature is essentially equal to the reference temperature.

20. The method of claim 17, comprising the further step of arranging the image sensor and the light source within a sealed common compartment.
